(19) United States
(12) Patent Application Publication
(10) Pub. No.: US 2024/0395394 A1
(43) Pub. Date: Nov. 28, 2024

(54) METHODS AND APPARATUS FOR RADIOABLATION TREATMENT AREA TARGETING AND GUIDANCE

(71) Applicant: VARIAN MEDICAL SYSTEMS, INC., Palo Alto, CA (US)

(72) Inventors: Jonas HONEGGER, Zurich (CH); Francesca ATTANASI, Baar (CH); Leigh JOHNSON, Chicago, IL (US); Andrea MORGAN, Seattle, WA (US)

(21) Appl. No.: 18/694,322

(22) PCT Filed: Sep. 22, 2022
(86) PCT No.: PCT/US2022/076835

§ 371 Date: Mar. 21, 2024

Related U.S. Application Data
(60) Provisional application No. 63/250,501, filed on Sep. 30, 2021.
(60) Provisional application No. 63/250,521, filed on Sep. 30, 2021.

(51) Int. Cl. G16H 30/40; G06T 7/11; G16H 20/40; G16H 30/20; G16H 50/20

(52) U.S. Cl. CPC G16H 30/40; G06T 7/11; G16H 20/40; G16H 30/20; G16H 50/20; G06T 2207/10081; G06T 2207/10088; G06T 2207/20081

(57) ABSTRACT

Systems (100) and methods (700, 800, 900) for target area recommendation and guidance during radioablation treatment planning are disclosed. In some examples. a computing device (104) receives image data (103) from one or more modalities for a patient. The computing device (104) determines a recommended target area for treatment based on the image data (103), and determines one or more corresponding segments of a segment model based on the recommended target area. Further, the computing device (104) displays the segment model identifying the determined one or more segments. and receives input data modifying the determined one or more segments. Based on the input data, the computing device (104) updates the one or more segments, and generates target definition data characterizing the updated one or more segments. The computing device (104) transmits the target definition data for treating the patient.

### CROSS-REFERENCE TO RELATED APPLICATIONS

This application claims priority to U.S. Provisional Application Ser. No. 63/250,501, filed on Sep. 30, 2021 and entitled “METHODS AND APPARATUS FOR RADIOABLATION TREATMENT AREA TARGETING AND GUIDANCE,” and to U.S. Provisional Application Ser. No. 63/250,521, filed on Sep. 30, 2021 and entitled “METHODS AND APPARATUS FOR RADIOABLATION TREATMENT AREA TARGETING AND GUIDANCE,” each of which is hereby incorporated by reference in its entirety.

### FIELD

Aspects of the present disclosure relate in general to medical diagnostic and treatment systems and, more particularly, to providing radioablation diagnostic, treatment planning, and delivery systems for treatment of conditions, such as cardiac arrhythmias.

### BACKGROUND

Various technologies can be employed to capture or image a patient's metabolic, electrical, and anatomical information. For example, positron emission tomography (PET) is a metabolic imaging technology that produces tomographic images representing the distribution of positron emitting isotopes within a body. Computed Tomography (CT) and Magnetic Resonance Imaging (MRI) are anatomical imaging technologies that create images using x-rays and magnetic fields respectively. Images from these exemplary technologies can be combined with one another to generate composite anatomical and functional images. For example, software systems, such as Velocity™ software from Varian Medical Systems, Inc., combine different types of images using an image fusion process to deform and/or register images to produce a combined image. Medical professionals, such as electrophysiologists and radiation oncologists, rely on these images to identify target areas for treatment.

For example, in cardiac radioablation, medical professionals work together to diagnose cardiac arrhythmias, identify regions for ablation, prescribe radiation treatment, and create radioablation treatment plans. An electrophysiologist may identify one or more regions or targets of a patient's heart for treatment of cardiac arrhythmias based on a patient's anatomy and electrophysiology. The electrophysiologist may, for example, rely on combined PET and cardiac CT images to define a target region for ablation. Once a target region is defined by the electrophysiologist, a radiation oncologist may prescribe radiation treatment including, for example, a number of fractions of radiation to be delivered, a radiation dose to be delivered to a target region, and a maximum dose to adjacent organs at risk. Further, a dosimetrist may create a radioablation treatment plan based on the prescribed radiation treatment. The radiation oncologist may also review and approve the treatment plan. In addition, the electrophysiologist may want to understand the location, size, and shape of the defined target region to confirm the target location as defined by the radioablation treatment plan is correct.

Properly identifying and defining the target region of a patient's organ for treatment is essential for developing and optimizing the treatment plan. For example, an over-inclusive target region may result in a defined target volume that includes areas that do not require treatment, while an under-inclusive target region may result in a defined target volume that fails to include areas that should be treated. As such, there are opportunities to improve radioablation treatment planning systems used by medical professionals, such as cardiac radioablation treatment systems used for cardiac radioablation treatment planning.

### SUMMARY

According to a first aspect of the invention, there is provided a system in accordance with claim 1.

According to a second aspect of the invention, there is provided a computer-implemented method in accordance with claim 16.

According to a third aspect of the invention, there is provided a non-transitory computer readable medium in accordance with claim 19.

Systems and methods for cardiac radioablation treatment and planning are disclosed. In some examples, a computing device receives image data for a patient. For example, the computing device may receive magnetic resonance (MR) image data, computed tomography (CT) image data, or positron emission tomography (PET) image data from an image scanning system. Based on the received image data, the computing device determines a recommended target area for treatment. In some examples, the computing device may also receive report data for the patient. The report data may characterize medical findings of the patient, such as a diagnosis of the patient. The computing device may determine the recommended target area for treatment based on the image data and the report data.

Further, the computing device receives an input identifying a change to the recommended target area for treatment. The computing device also determines if any of one or more rules are violated based on the change to the recommended target area for treatment. The computing device also provides for display an indication of whether the change is accepted based on determining if any of the one or more rules are violated. For example, if no rules are violated, the computing device may update the recommended target area for treatment based on the change, and provide for display the updated recommended target area. If, however, one or more rules are violated, the computing device may provide for display an error message.

In some examples, a system includes a database, and a computing device communicatively coupled to the database. The computing device is configured to receive image data for an organ of a patient. The computing device is also configured to determine a recommended target area of the organ for treatment based on the image data. Further, the computing device is configured to generate recommended target data characterizing the recommended target area of the organ. The computing device is also configured to store the recommended target data in the database.

In some examples, a computing device is configured to receive a first input identifying a change to a recommended target area for treatment. The computing device is also configured to determine if a first rule is violated based on the change to the recommended target area for treatment. Further, the computing device is configured to provide for display an indication of whether the change is accepted based on determining if any of the one or more rules are violated.

In some examples, a computing device is configured to receive image data for a patient. The computing device is also configured to determine a scar location of an organ based on the image data. Further, the computing device is also configured to determine a segment of a plurality of segments of a model of the organ based on the scar location. The computing device is also configured to display the model with an identification of the determined segment. For example, the computing device may display the determined segment in one color, and the remaining segments of the plurality of segments in another color. In some examples, the computing device is configured to display the model with an overlay of the image data. In some examples, the computing device is configured to display the image data with an overlay of the model.

In some examples, a computing device is configured to receive image data for a patient. The computing device is also configured to determine a scar location of an organ based on the image data. Further, the computing device is configured to determine healthy portions of the organ based on the scar location. The computing device is also configured to display a model of the organ with an identification of the scar location and the healthy portions. For example, the computing device may display the scar location of the organ in one color, and the healthy portions of the organ in another color.

In some examples, a computer-implemented method includes receiving image data for a patient. The method also includes determining, based on the received image data, a recommended target area for treatment. In some examples, the method also includes receiving report data for the patient. The method then includes determining the recommended target area for treatment based on the image data and the report data.

Further, the method includes receiving an input identifying a change to the recommended target area for treatment. The method also includes determining if any of one or more rules are violated based on the change to the recommended target area for treatment. The method further includes providing for display an indication of whether the change is accepted based on determining if any of the one or more rules are violated.

In some examples, a method includes receiving image data for an organ of a patient. The method also includes determining a recommended target area of the organ for treatment based on the image data. Further, the method includes generating recommended target data characterizing the recommended target area of the organ. The method also includes the recommended target data in a database.

In some examples, a method includes receiving a first input identifying a change to a recommended target area for treatment. The method also includes determining if a first rule is violated based on the change to the recommended target area for treatment. Further, the method includes providing for display an indication of whether the change is accepted based on determining if any of the one or more rules are violated.

In some examples, a computer-implemented method includes receiving image data for a patient. The method also includes determining a scar location of an organ based on the image data. Further, the method includes determining a segment of a plurality of segments of a model of the organ based on the scar location. The method also includes displaying the model with an identification of the determined segment. In some examples, the method includes displaying the model with an overlay of the image data. In some examples, the method includes displaying the image data with an overlay of the model.

In some examples, a computer-implemented method includes receiving image data for a patient. The method includes determining a scar location of an organ based on the image data. Further, the method includes determining healthy portions of the organ based on the scar location. The method also includes displaying a model of the organ with an identification of the scar location and the healthy portions.

In some examples, a non-transitory computer readable medium storing instructions that, when executed by at least one processor, cause the at least one processor to perform operations including receiving image data for a patient. The operations also include determining, based on the received image data, a recommended target area for treatment. In some examples, the operations also include receiving report data for the patient. The operations then include determining the recommended target area for treatment based on the image data and the report data.

Further, the operations include receiving an input identifying a change to the recommended target area for treatment. The operations also include determining if any of one or more rules are violated based on the change to the recommended target area for treatment. The operations further include providing for display an indication of whether the change is accepted based on determining if any of the one or more rules are violated.

In some examples, a non-transitory computer readable medium storing instructions that, when executed by at least one processor, cause the at least one processor to perform operations including receiving image data for an organ of a patient. The operations also include determining a recommended target area of the organ for treatment based on the image data. Further, the operations include generating recommended target data characterizing the recommended target area of the organ. The operations also include the recommended target data in a database.

In some examples, a non-transitory computer readable medium storing instructions that, when executed by at least one processor, cause the at least one processor to perform operations including receiving a first input identifying a change to a recommended target area for treatment. The operations also include determining if a first rule is violated based on the change to the recommended target area for treatment. Further, the operations include providing for display an indication of whether the change is accepted based on determining if any of the one or more rules are violated.

In some examples, a non-transitory computer readable medium storing instructions that, when executed by at least one processor, cause the at least one processor to perform operations including receiving image data for a patient. The operations also include determining a scar location of an organ based on the image data. Further, the operations include determining a segment of a plurality of segments of a model of the organ based on the scar location. The operations also include displaying the model with an identification of the determined segment. In some examples, the operations include displaying the model with an overlay of the image data. In some examples, the operations include displaying the image data with an overlay of the model.

In some examples, a non-transitory computer readable medium storing instructions that, when executed by at least one processor, cause the at least one processor to perform operations including receiving image data for a patient. The operations include determining a scar location of an organ based on the image data. Further, the operations include determining healthy portions of the organ based on the scar location. The operations also include displaying a model of the organ with an identification of the scar location and the healthy portions.

In some examples, a computer-implemented method includes a means for receiving image data for a patient. The method also includes a means for determining, based on the received image data, a recommended target area for treatment. In some examples, the method also includes a means for receiving report data for the patient. The method then includes a means for determining the recommended target area for treatment based on the image data and the report data.

Further, the method includes a means for receiving an input identifying a change to the recommended target area for treatment. The method also includes a means for determining if any of one or more rules are violated based on the change to the recommended target area for treatment. The method further includes a means for providing for display an indication of whether the change is accepted based on determining if any of the one or more rules are violated.

In some examples, a computer-implemented method includes a means for receiving image data for an organ of a patient. The method also includes a means for determining a recommended target area of the organ for treatment based on the image data. Further, the method includes a means for generating recommended target data characterizing the recommended target area of the organ. The method also includes a means for storing the recommended target data in a database.

In some examples, a computer-implemented method includes a means for receiving a first input identifying a change to a recommended target area for treatment. The method also includes a means for determining if a first rule is violated based on the change to the recommended target area for treatment. Further, the method includes a means for providing for display an indication of whether the change is accepted based on determining if any of the one or more rules are violated.

In some examples, a computer-implemented method includes a means for receiving image data for a patient. The method also includes a means for determining a scar location of an organ based on the image data. Further, the method includes a means for determining a segment of a plurality of segments of a model of the organ based on the scar location. The method also includes a means for displaying the model with an identification of the determined segment. In some examples, the method includes a means for displaying the model with an overlay of the image data. In some examples, the method includes a means for displaying the image data with an overlay of the model.

In some examples, a computer-implemented method includes a means for receiving image data for a patient. The method includes a means for determining a scar location of an organ based on the image data. Further, the method includes a means for determining healthy portions of the organ based on the scar location. The method also includes a means for displaying a model of the organ with an identification of the scar location and the healthy portions.

### BRIEF DESCRIPTION OF THE DRAWINGS

The features and advantages of the present disclosures will be more fully disclosed in, or rendered obvious by the following detailed descriptions of example embodiments. The detailed descriptions of the example embodiments are to be considered together with the accompanying drawings wherein like numbers refer to like parts and further wherein:

FIG. 1 illustrates a cardiac radioablation targeting system, in accordance with some embodiments;

FIG. 2 illustrates a block diagram of a target recommendation computing device, in accordance with some embodiments;

FIG. 3 illustrates exemplary portions of the cardiac radioablation treatment system of FIG. 1, in accordance with some embodiments;

FIG. 4 illustrates a 2-dimensional 17 segment model of a heart, in accordance with some embodiments;

FIGS. 5A, 5B, and 5C illustrate portions of a graphical user interface for recommending and selecting target areas within a model, in accordance with some embodiments;

FIGS. 6A, 6B, 6C, 6D, and 6E illustrate portions of a graphical user interface for recommending and selecting target areas within a 3-dimensional image, in accordance with some embodiments;

FIG. 7 is a flowchart of an example method to recommend and adjust target areas for treatment, in accordance with some embodiments;

FIG. 8 is a flowchart of an example method to generate and display a digital model of a scar location of an organ, in accordance with some embodiments; and

FIG. 9 is a flowchart of an example method to generate and display a digital model identifying scar locations and healthy locations of an organ, in accordance with some embodiments.

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

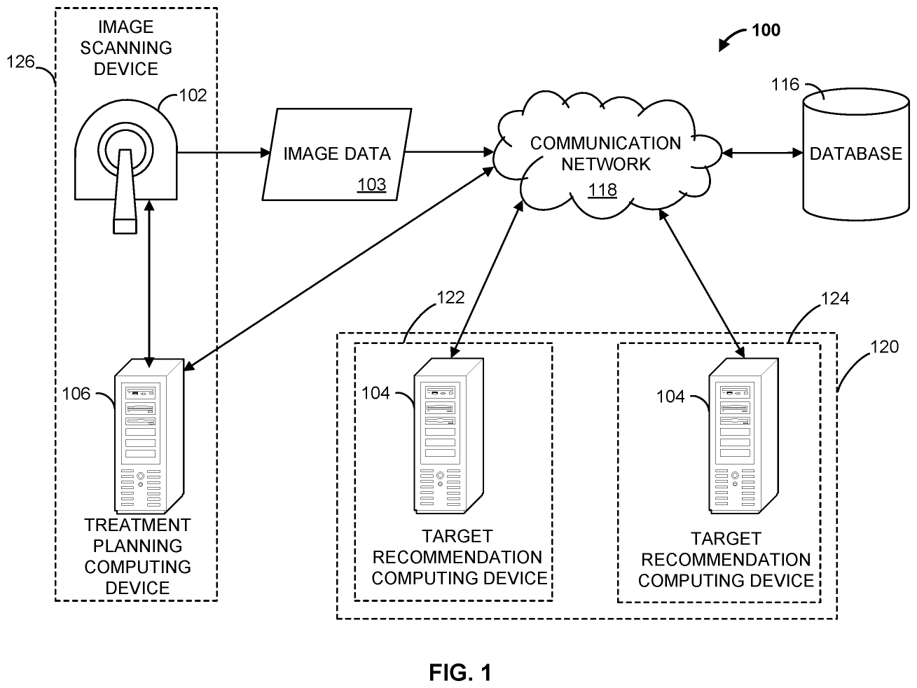

Turning to the drawings, FIG. 1 illustrates a block diagram of a cardiac radioablation targeting system 100 that includes an imaging device 102, a treatment planning computing device 106, one or more target recommendation computing devices 104, and a database 116 communicatively coupled over communication network 118. Imaging device 102 may be, for example, a CT scanner, an MR scanner, a PET scanner, an electrophysiologic imaging device, an ECG, or an ECG imager. In some examples, imaging device 102 may be PET/CT scanner or a PET/MR scanner. In some examples, imaging device 102 and treatment planning computing device 106 may be part of a radioablation treatment system 126 that allows for radioablation treatment to a patient. For example, radioablation treatment system 126 may allow for the delivery of defined doses to one or more treatment areas of the patient.

Each target recommendation computing device 104 and treatment planning computing device 106 can be any suitable computing device that includes any suitable hardware or hardware and software combination for processing data. For example, each can include one or more processors, one or more field-programmable gate arrays (FPGAs), one or more application-specific integrated circuits (ASICs), one or more state machines, digital circuitry, or any other suitable circuitry. In addition, each can transmit data to, and receive data from, communication network 118. For example, each of target recommendation computing device 104 and treatment planning computing device 106 can be a server such as a cloud-based server, a computer, a laptop, a mobile device, a workstation, or any other suitable computing device.

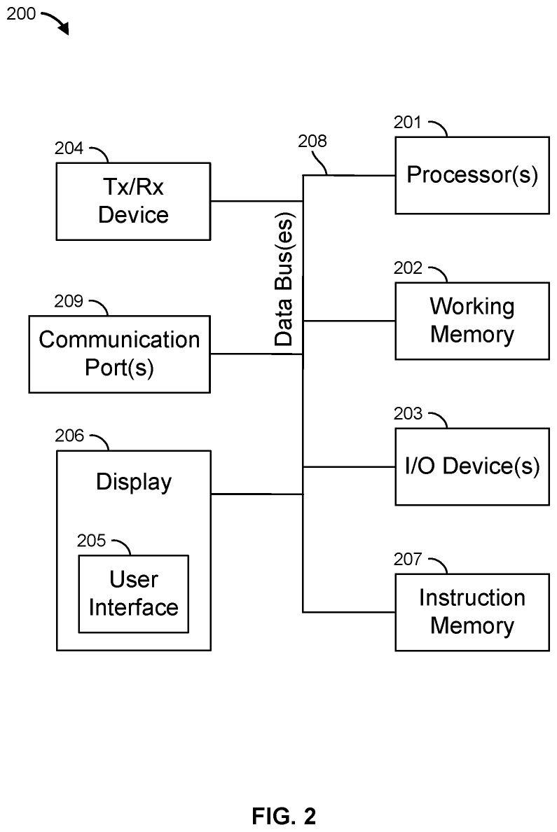

For example, FIG. 2 illustrates a computing device 200, which may be an example of each of target recommendation computing device 104 and treatment planning computing device 106. Computing device 200 includes one or more processors 201, working memory 202, one or more input-output (I/O) devices 203, instruction memory 207, a transceiver 204, one or more communication ports 209, and a display 206, all operatively coupled to one or more data buses 208. Data buses 208 allow for communication among the various devices. Data buses 208 can include wired, or wireless, communication channels.

Processors 201 can include one or more distinct processors, each having one or more cores. Each of the distinct processors can have the same or different structure. Processors 201 can include one or more central processing units (CPUs), one or more graphics processing units (GPUs), application specific integrated circuits (ASICs), digital signal processors (DSPs), and the like.

Instruction memory 207 can store instructions that can be accessed (e.g., read) and executed by processors 201. For example, instruction memory 207 can be a non-transitory, computer-readable storage medium such as a read-only memory (ROM), an electrically erasable programmable read-only memory (EEPROM), flash memory, a removable disk, CD-ROM, any non-volatile memory, or any other suitable memory. Processors 201 can be configured to perform a certain function or operation by executing code, stored on instruction memory 207, embodying the function or operation. For example, processors 201 can be configured to execute code stored in instruction memory 207 to perform one or more of any function, method, or operation disclosed herein.

Additionally processors 201 can store data to, and read data from, working memory 202. For example, processors 201 can store a working set of instructions to working memory 202, such as instructions loaded from instruction memory 207. Processors 201 can also use working memory 202 to store dynamic data created during the operation of computing device 200. Working memory 202 can be a random access memory (RAM) such as a static random access memory (SRAM) or dynamic random access memory (DRAM), or any other suitable memory.

Input-output devices 203 can include any suitable device that allows for data input or output. For example, input-output devices 203 can include one or more of a keyboard, a touchpad, a mouse, a stylus, a touchscreen, a physical button, a speaker, a microphone, or any other suitable input or output device.

Communication port(s) 209 can include, for example, a serial port such as a universal asynchronous receiver/transmitter (UART) connection, a Universal Serial Bus (USB) connection, or any other suitable communication port or connection. In some examples, communication port(s) 209 allows for the programming of executable instructions in instruction memory 207. In some examples, communication port(s) 209 allow for the transfer (e.g., uploading or downloading) of data, such as image data.

Display 206 can be any suitable display, such as a 3D viewer or a monitor. Display 206 can display user interface 205. User interfaces 205 can enable user interaction with computing device 200. For example, user interface 205 can be a user interface for an application that allows a user (e.g., a medical professional) to view or manipulate models to define a target region of treatment for a patient as described herein. In some examples, the user can interact with user interface 205 by engaging input-output devices 203. In some examples, display 206 can be a touchscreen, where user interface 205 is displayed on the touchscreen. In some examples, display 206 displays images of scanned image data (e.g., image slices).

Transceiver 204 allows for communication with a network, such as the communication network 118 of FIG. 1. For example, if communication network 118 of FIG. 1 is a cellular network, transceiver 204 is configured to allow communications with the cellular network. In some examples, transceiver 204 is selected based on the type of communication network 118 radioablation targeting computing device 200 will be operating in. Processor(s) 201 is operable to receive data from, or send data to, a network, such as communication network 118 of FIG. 1, via transceiver 204.

Referring back to FIG. 1, database 116 can be a remote storage device (e.g., including non-volatile memory), such as a cloud-based server, a disk (e.g., a hard disk), a memory device on another application server, a networked computer, or any other suitable remote storage. In some examples, database 116 can be a local storage device, such as a hard drive, a non-volatile memory, or a USB stick, to one or more of target recommendation computing device 104 and treatment planning computing device 106.

Communication network 118 can be a WiFi® network, a cellular network such as a 3GPPR network, a Bluetooth® network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. Communication network 118 can provide access to, for example, the Internet.

Imaging device 102 is operable to scan images, such as images of a patient's organs, and provide image data 103 (e.g., measurement data) identifying and characterizing the scanned images to communication network 118. Alternatively, imaging device 102 is operable to acquire electrical imaging such as cardiac ECG images. For example, imaging device 102 may scan a patient's structure (e.g., organ), and may transmit image data 103 identifying one or more slices of a 3D volume of the scanned structure over communication network 118 to one or more of target recommendation computing device 104 and treatment planning computing device 106. In some examples, imaging device 102 stores image data 103 in database 116, and one or more of target recommendation computing device 104 and treatment planning computing device 106 may retrieve the image data 103 from database 116.

In some examples, target recommendation computing device 104 is operable to communicate with treatment planning computing device 106 over communication network 118. In some examples, target recommendation computing device 104 and treatment planning computing device 106 communicate with each other via database 116 (e.g., by storing and retrieving data from database 116). In some examples, one or more target recommendation computing devices 104 and one or more treatment planning computing devices 106 are part of a cloud-based network that allows for the sharing of resources and communication with each device.

In some examples, one or more target recommendation computing devices 104 are located in a first area 122 of a medical facility 120, while one or more target recommendation computing devices 104 are located in a second area 124 of the medical facility 120. As such, cardiac radioablation targeting system 100 allows multiple electrophysiologists (EPs) to collaborate to finalize the target area. For example, one EP may operate a first target recommendation computing device 104 in a first medical facility 122, and a second EP may operate a second target recommendation computing device 104 in a second medical facility 124. First target recommendation computing device 104 and second target recommendation computing device 104 may communicate over communication network 118, such as by transmitting and receiving data related to (e.g., defining) the target area (e.g., a proposed target area). Each EP may operate the corresponding target recommendation computing device 104 to adjust the target area, and may finalize the target area once both EPs are in agreement of the target area.

### Target Area Recommendation

As described herein, target recommendation computing device 104 may execute an application that causes the generation of a user interface (e.g., user interface 205) which may be displayed to a medical professional, such as an EP. The executed application may assist the medical professional in defining a target area of a patient for treatment. For example, an electrophysiologist (EP) may operate target recommendation computing device 104 to define a target region of treatment for a patient. Target recommendation computing device 104 can recommend a target region (e.g., an initial target region) for treatment based on patient data, such as image data 103 captured by image scanning device 102 for the patient.

To determine the initial target region, target recommendation computing device 104 may perform one or more processes that analyze the image data, and that identify the initial target region. The initial target region may include, for example, a scar location. For example, target recommendation computing device 104 may apply one or more machine learning processes (e.g., models, algorithms) to the image data to define the initial target region. The machine learning processes may be trained using supervised or unsupervised learning and/or based on features generated from historical image scans. For example, a first machine learning process may be trained on features generated from CT data characterizing previous CT scans, a second machine learning process may be trained on features generated from MR data characterizing previous MR scans, and a third machine learning process may be trained on features generated from PET data characterizing previous PET scans.

Further, and based on the scar location, target recommendation computing device 104 may identify healthy portions of the organ. In some examples, target recommendation computing device 104 determines the healthy portions based on applying one or more rules to a determined location (e.g., 3-dimensional location) of the scar location within the organ. For example, target recommendation computing device 104 may identify areas of the organ that are at least a minimum distance from the scar location as healthy portions of the organ.

In some examples, target recommendation computing device 104 obtains electrocardiogram (EKG) data for the patient, and determines the initial target region based on the EKG data. For example, target recommendation computing device 104 may apply a trained machine learning process to the EKG data to determine the initial target region as described herein. In some examples, the trained machine learning process is applied to one or more of MR image data, CT image data, PET image data, and EKG image data to determine the initial target region.

In some examples, target recommendation computing device 104 determines the initial target area based on report data characterizing medical professional findings and/or diagnosis of the patient. For example, database 116 may store report data that characterizes medical reports. The medical reports may include characterizations of conditions, areas of concern, location information (e.g., location of organ areas for scarring), health information, medical professional findings, diagnosis of patients, or any other medical information. Target recommendation computing device 104 may obtain the report data for the patient from database 116, and apply a text extracting process to the report data to identify text. Further, target recommendation computing device 104 may apply a trained machine learning process to the text data as well as, in some examples, to the image data, to determine the initial target area.

In some examples, the report data characterizes audio, such as the voice of one or more medical professionals. Target recommendation computing device 104 may apply one or more voice-to-text models to the report data to extract text data. For example, target recommendation computing device 104 may apply a speech recognizer algorithm to the report data to extract the text.

In some examples, target recommendation computing device 104 applies one or more rules to the text data and/or the image data to determine the initial target area, which may include a scar location. For example, database 116 may store rule data characterizing the one or more rules, which may have been configured by one or more medical professionals. A rule may associate, for example, one or more words of text with a first target area, and one or more different words with a second target area. Target recommendation computing device 104 may determine, for example, if the text extracted from the report data includes any of the one or more words associated with the first target area, or the one or more different words associated with the second target area. Based on any corresponding words, target recommendation computing device 104 may determine the initial target area as either the first target area or the second target area. In some examples, target recommendation computing device 104 determines the initial target area to be the one with the most corresponding words. In some examples, target recommendation computing device 104 applies one or more rules to the text extracted from report data to determine the healthy portions.

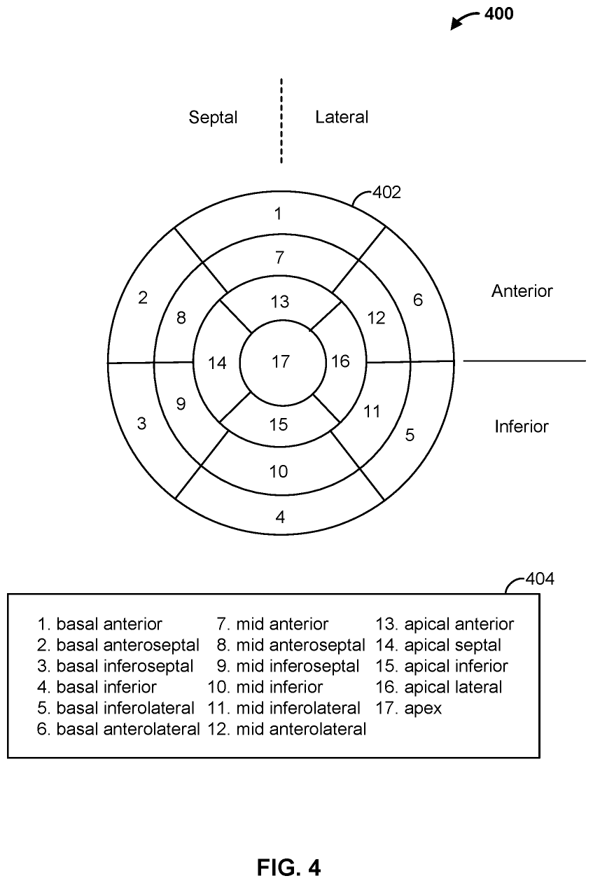

Further, in some examples, target recommendation computing device 104 may associate the initial target area with a portion of an organ's model, such as with a particular segment of an organ's segment model. For example, FIG. 4 illustrates a 17-segment model 402 of a heart's ventricle which may be displayed, for example, by a GUI 400. Each of the 17segments correspond with a portion of the heart ventricle, as identified by model key 404. For example, segment one corresponds to the basal anterior portion of a heart ventricle, while segment 17 corresponds to the apex portion of the heart ventricle. Target recommendation computing device 104 may determine a segment of the 17-segment model 402 corresponding to the initial target area. In some examples, target recommendation computing device 104 determines the segment based on a relative location of the initial target area to a portion of the heart, such as the apex. For example, target recommendation computing device 104 may determine a distance and direction from the apex of the heart to the initial target area of the heart, and based on the distance and direction, determine the corresponding segment. In some examples, target recommendation computing device 104 determines the corresponding segment based on one or more rules. The rules may identify a correlation, for example, of one or more words of text (e.g., extracted from the report data) with a particular segment.

In some examples, target recommendation computing device 104 determines a segment of a model based on image data received for each of a plurality of imaging technologies. For example, target recommendation computing device 104 may obtain from database 116 CT image data, MR image data, and PET image data for a patient. Target recommendation computing device 104 may determine a segment of a segment model based on each of the CT image data, MR image data, and PET image data for the patient. Further, target recommendation computing device 104 may determine whether the determined segments are the same (e.g., match). If they are the same, target recommendation computing device 104 may generate segment data characterizing the determined segment, and may store the segment data within database 116. If the determined segments are not the same, target recommendation computing device 104 may apply one or more additional rules to the determined segments to generate the segment data. For example, target recommendation computing device 104 may determine a number of times a particular segment was determined, and generate segment data identifying the most determined segment.

In some instances, target recommendation computing device 104 may apply a weighting to each of the determined segments, and generate the target data based on the weighted segments. For example, target recommendation computing device 104 may apply a 40% weighting to PET image data, and 30% weighting to each of the segments determined based on MR image data and CT image data. If the three determined weightings are different, target recommendation computing device 104 may select the segment determined based on PET image data. If the determined segments based on MR image data and CT image data are the same, target recommendation computing device 104 may select that determined segment over the segment determined on PET image data (e.g., as 60% is greater than 40%).

Target recommendation computing device 104 may provide the initial target region and/or determined segments of a model for display. For example, target recommendation computing device 104 may reconstruct an image based on received image data, where the reconstructed image identifies the initial target region. The reconstructed image may be, for example, a 2-dimensional or 3-dimensional image. For instance, the initial target region may be in a different color that the other portions of the organ. In other examples, target recommendation computing device 104 may outline, highlight, or hash the initial target region within the reconstructed image, or may identify the initial target region within the reconstructed image in any other suitable manner. Further, target recommendation computing device 104 may provide the reconstructed image for display. As such, a medical professional, such as an EP, can easily identify the initial target region.

In some examples, target recommendation computing device 104, additionally or alternately, displays the segment model described herein. In some examples, target recommendation computing device 104 may outline, highlight, or hash the determined segment, or may identify the determined segment in any other suitable manner. In some instances, target recommendation computing device 104 provides for display the reconstructed image overlaid over the segment model. For example, the EP may view the scarred areas as identified in the reconstructed image over a 17-segment heart ventricle model. In other instances, target recommendation computing device 104 provides for display the segment model overlaid over the reconstructed image.

Further, in some examples, target recommendation computing device 104 displays the segment model with an identification of the scar location and the healthy portions of the organ. For example, target recommendation computing device 104 may display a segment model with segments corresponding to the scar location displayed differently than segments corresponding to healthy portions of the organ. For example, the segments corresponding to the scar location may be displayed in a different color, or highlighted or hashed differently, than the segments corresponding to the health portions of the organ.

### Target Area Adjustment Guidance

Target recommendation computing device 104 may also allow medical professionals, such as the EP, to modify, change, or update target regions, such as a recommended target region as described herein. Once finalized, target recommendation computing device 104 may generate target definition data identifying the finalized target region for a patient, and can transmit the target definition data to treatment planning computing device 106. A medical professional such as a radiation oncologist may operate treatment planning computing device 106 to deliver treatment to the patient via imaging device 102 to the area of the patient defined by the target definition data. In some examples, the target definition region is integrated into a radioablation treatment plan for treating the patient.

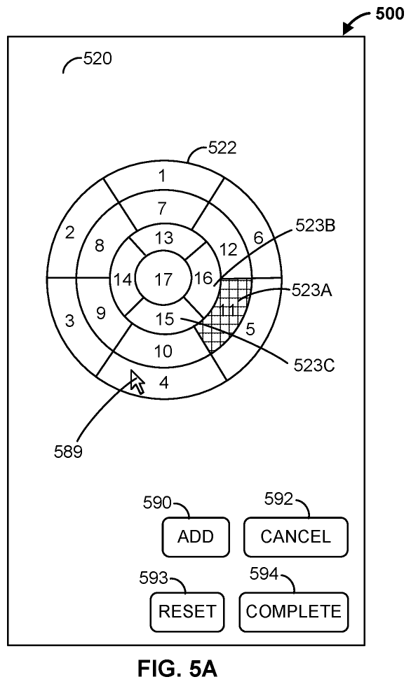

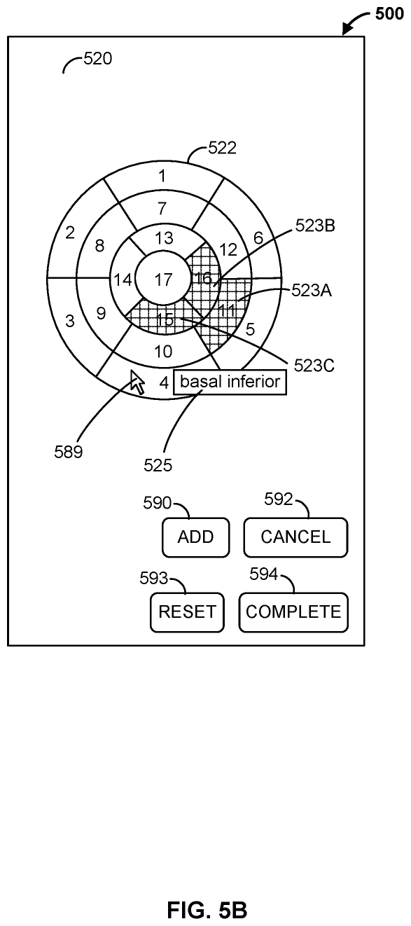

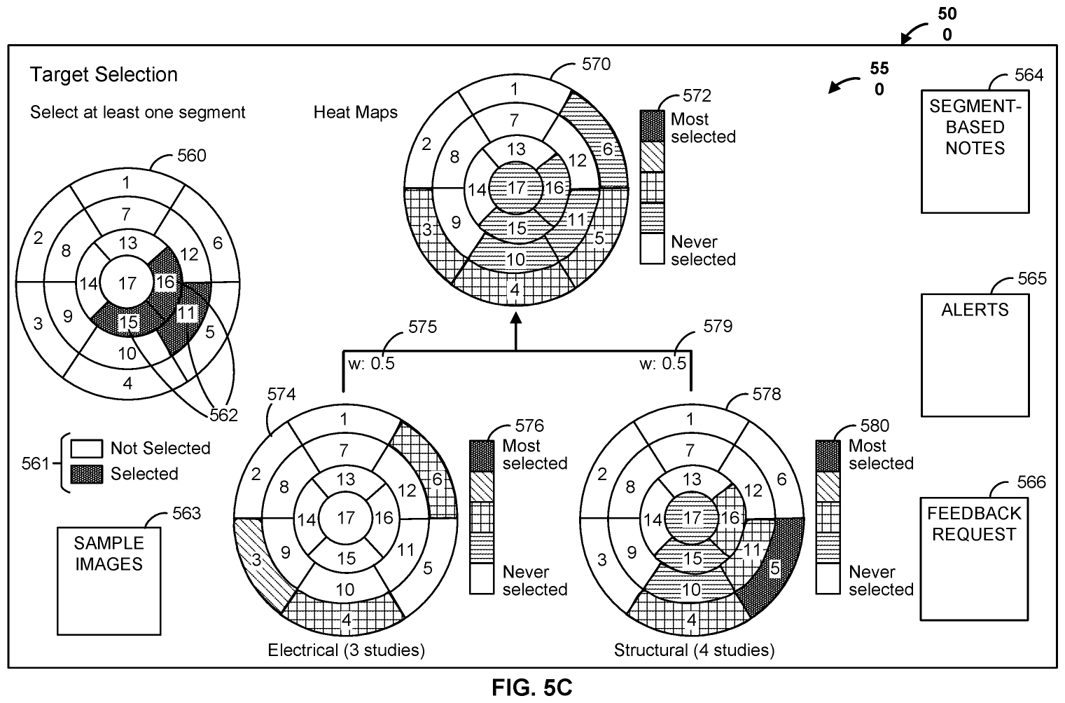

The application executed by target recommendation computing device 104 may facilitate the modifications, changes, or updates to the recommended target region (e.g., the initial target region). For example, FIG. 5A illustrates a graphical user interface (GUI) 520 of a display interface 500 that includes an interactive model 522. In this example, interactive model 522 is a 17-segment model representing segments of a heart's ventricle. The medical professional may select, or deselect, one or more segments of the interactive model 522, which may correspond to areas for treatment. When first displayed to the medical professional, the interactive model 522 may identify recommended segments corresponding to recommended areas for treatment (i.e., the recommended target region). In some examples, however, interactive model 522 does not identify any recommended segments. In this example, the recommended segments include a first segment 523A (e.g., segment 11). Assume, however, that the medical professional would like to mark additional segments for treatment. The medical professional may simply select those additional segments within the interactive model 522 using cursor 589 (e.g., using I/O device 203).

For example, and as illustrated in FIG. 5B, the medical professional may select a second segment 523B (e.g., segment 16), and a third segment 523C (e.g., segment 15). Likewise, the medical professional may deselect any selected segments. In some examples, when a cursor 589 is placed over a segment (e.g., segment 4), GUI 520 displays the name of the segment (e.g., via a pop-up window). In this example, cursor 589 appears over segment 4 of interactive model 522, and in response GUI 520 displays name box 525 identifying segment 4 as the “basal inferior” portion of a heart's ventricle.

To add a segment to the recommended target region, the medical professional may click on add icon 590. In response, target recommendation computing device 104 generates data identifying and characterizing the updated target region, and stores the generated data in a data repository, such as within database 116. If the medical professional would like to start over and not save the segment selections and de-selections, the medical professional may click on the cancel icon 592, which results in the clearance of any selected or de-selected segments since the add icon 590 was last clicked. In some examples, GUI 520 includes a reset icon 593 that, if selected, clears any modifications made by the medical professional and results in the initial recommended segments (e.g., as originally recommended).

Once the medical professional is complete with any changes to the recommended segments (e.g., assuming any changes are even made), the medical professional may select the complete icon 594. Based upon the selection of complete icon 594, the executed application may display GUI 550 as illustrated in FIG. 5C. GUI 550 includes interactive model 560, which identifies the selected segments 562 (e.g., as selected in GUI 520). The selected segments 562 may be identified according to any suitable method and, in this example, as indicated by key 561. For example, selected segments may appear in a different color than unselected segments. In some examples, selected segments may be highlighted or hashed (e.g., differently than unselected segments), and/or identified by segment number. In addition, the medical professional may adjust the selected segments by selecting and/or deselecting segments within interactive model 560, as described with respect to interactive model 522, for example.

Further, GUI 550 may include one or more study category maps (e.g., “heat maps”) that characterize the corresponding patient's previous studies and treatments. For example, GUI 550 includes an electrical map 574, a structural map 578, and a combined map 570. Electrical map 574 may characterize previous electrical studies and treatments, such as those based on EKG results. Key 576, which corresponds to electrical map 574, provides an indication of the relative number of times each segment has been previously selected for an electrical study and/or treatment for the patient. For example, key 576 may indicate the most selected segment(s) and the least selected segment(s). In some examples, each category indicated by key 576 may correspond to a range of a number of times each segment was selected (e.g., a first category 0 times, a second category 1 time, a third category 2-4 times, a fourth category 5-7 times, and a fifth category 8 and above times). Key 576 may provide the indications using any suitable method, such as employing a hashing method or varying the display colors of the segments. In this example, segment 3 was previously selected more often than segments 4 and 6, for instance. Similarly, structural map 578 may characterize previous imaging studies and treatments, such as those based on CT, MR, or PET imaging. Key 580, which corresponds to structural map 578, provides an indication of the relative number of times each segment was previously selected for an imaging study and/or treatment for the patient. In this example, segment 5 was previously selected more often than any other segment.

Combined map 570 is based on the relative number of times segments were previously selected in the electrical and structural studies characterized by the electrical map 574 and the structural map 578. For example, target recommendation computing device 104 may determine the total number of times each segment has been selected between electrical and structural studies, and determine how often each segment was selected relative to others. Key 572, which corresponds to combined map 570, provides an indication of the relative number of times each segment was previously selected for any study and/or treatment for the patient. For example, key 572 may indicate the most selected segment(s) and the least selected segment(s). In some examples, each category indicated by key 572 may correspond to a range of a number of times each segment was selected. Key 572 may provide the indications using any suitable method, such as employing a hashing method or varying the display colors of the segments.

In some examples, target recommendation computing device 104 may apply a weight to the number of times each segment was selected for a previous type of study. For instance, target recommendation computing device 104 may weight structural studies more heavily than electrical studies, or vice-versa. Based on applying the weights, target recommendation computing device 104 determines how often each segment was selected relative to others. In this example, first weight 575 is applied to electrical studies, while second weight 579 is applied to structural studies. Although in FIG. 5C the first weight 575 and the second weight 579 are the same (e.g.,.5), they may differ. Indeed, in some examples, GUI 550 allows the medical professional to edit each of the first weight 575 and the second weight 579.

By providing the study category maps (e.g., electrical map 574, structural map 578, and combined map 570), GUI 550 provides additional information to the medical professional to assure the best suited segments are selected for treatment.

Additionally, GUI 550 may include a sample images 563 portion, which may display image scans of the patient and/or image scans of others that have had a similar condition and/or treatment. For instance, target recommendation computing device 104 may determine one or more previous patients that have had a similar condition and/or treatment, and obtain image data 103 for the patient and the one or more previous patients from database 116. Further, target recommendation computing device 104 may reconstruct the images based on the obtained image data, and display the images within the sample images 563 portion of GUI 550.

GUI 550 may further display segment-based notes 564, which may include text previously determined and provided for each selected segment. For instance, because in interactive model 560 segments 11, 15, and 16 are selected, any notes for those segments may appear within the segment-based notes 564 portion of GUI 550. The notes may include text pre-approved by one or more medical professionals, for example. In some examples, segment-based notes 564 allows a medical professional to enter in additional information (e.g., via I/O device 203).

GUI 550 further includes an alerts 565 portion, which may provide alerts (e.g., warnings, recommended checks, advice, etc.) based on the selected segments. For example, target recommendation computing device 104 may generate the alerts based on the application of one or more rules or, in some examples, on the application of one or more machine learning processes to image data and/or report data for the patient, as described herein. For example, target recommendation computing device 104 may cause the display of an alert if, based on previous studies of the patient, target recommendation computing device 104 determines a scar location has been located in a particular segment over a threshold amount or percentage of times (e.g., 75%), but that segment is not currently selected (e.g., within interactive model 560). As another example, target recommendation computing device 104 may cause the display of an alert if target recommendation computing device 104 determines that more than a threshold amount of segments are selected (e.g., 3). As yet another example, target recommendation computing device 104 may cause the display of an alert if target recommendation computing device 104 determines that a particular combination of segments is, or is not, selected (e.g., when segments 3 and 7 are selected; when segment 15 is selected but segment 10 is not; etc.). Rules such as these may be user-defined rules, and may be based on an agreement between one or more medical professionals (e.g., a consortium of medical professionals agreeing to best practices).

Other exemplary rules may include determining that SCAR SEGMENTS are selected at or adjacent to a ventricle (VT) EXIT SITE, with the goal of avoiding healthy tissue. Another rule may include limiting the number of TARGET segments based on the number of induced VTs. For example, a rule may allow 1-2 TARGET segments for 1 induced VT 1-4 TARGET segments for 2 induced VTs, and 1-6 TARGET segments for 3 or more induced VTs. In some examples, a rule may specify the maximum number of TARGET segments selected, such as six.

Additionally, GUI 550 includes a feedback request 566 portion, which allows a medical professional to seek input (e.g., opinions) of other medical professionals. For example, a medical professional may provide input to the feedback request 566 portion of GUI 550, and target recommendation computing device 104 may transmit the request to one or more other computing devices, such as another target recommendation computing device 104. In some examples, the request is transmitted to one or more predetermined computing devices. In some examples, the feedback request portion includes a menu (e.g., a drop-down menu) that allows for the selection of one or more medical professionals to transmit the request to. A receiving computing device may display the request, allow a medical professional to provide a response, and may further transmit the response back to the target recommendation computing device 104 that sent the request. Upon receiving the response, the target recommendation computing device 104 may display the response within the feedback request portion 566.

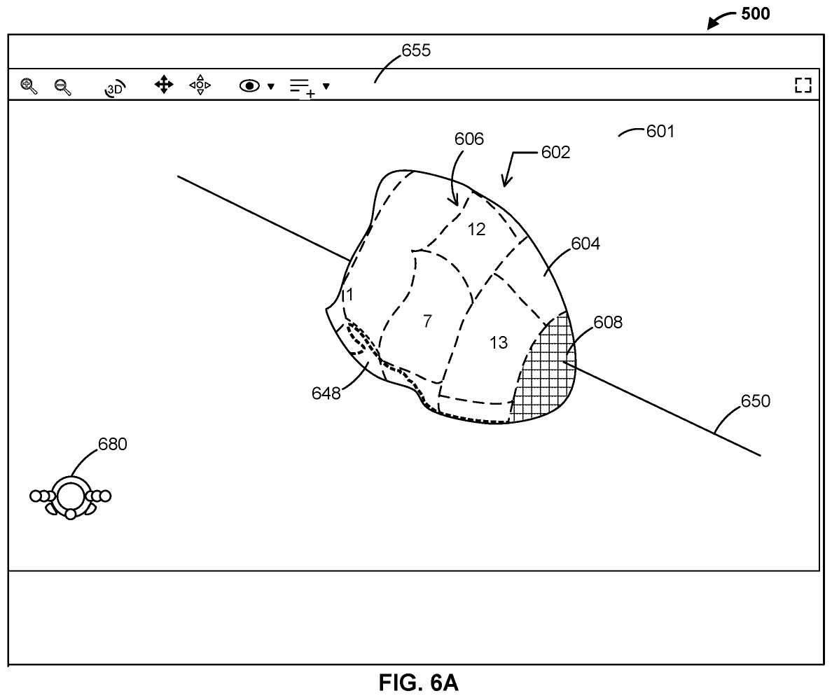

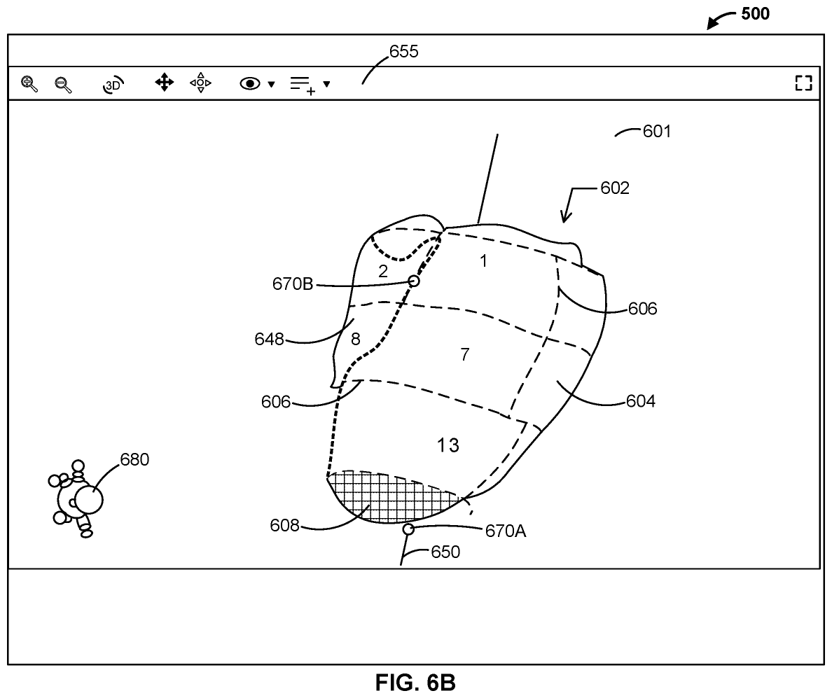

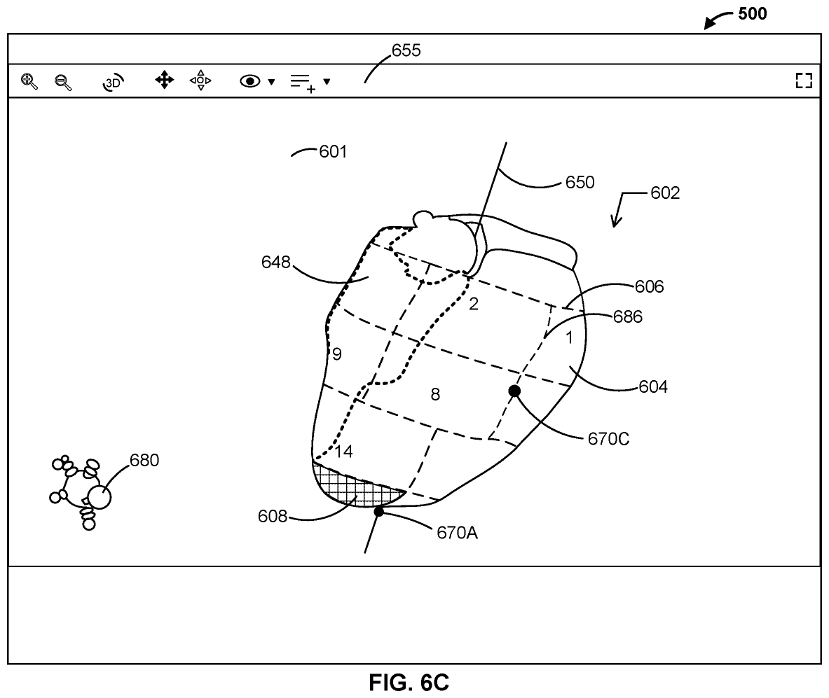

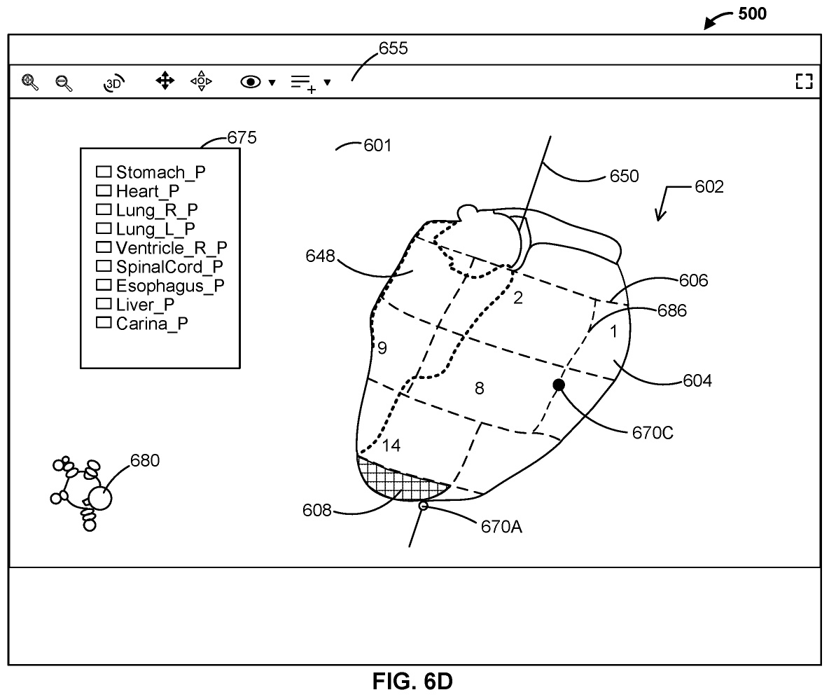

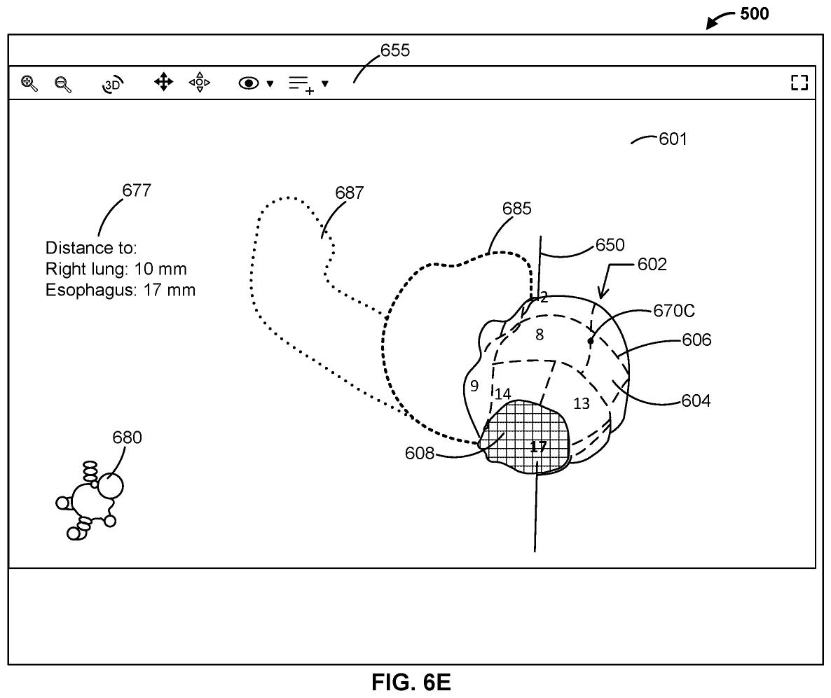

FIG. 6A illustrates an alignment GUI 601 of display interface 500 that can be generated by, for example, target recommendation computing device 104. Display interface 500 includes a 3D structure image 602 that includes a 3D segment model 606 superimposed onto scanned image 604. Target recommendation computing device 104 may generate the 3D structure image 602 based on image data (e.g., image data 103) for a patient and an interactive model, such as interactive model 522 or interactive model 560.

3D segment model 606 may be a 3D segment model of a heart's ventricle, for example. Scanned image 604 may be an image scanned by image scanning device 102, such as a 3D volume of a scanned structure of the patient. 3D structure image 602 also includes a target region map 648, which defines a target region for treatment for the patient. The target region map 648 may correspond to one or more selected target areas of an interactive model, such as first, second, and third segments 523A, 523B, 523C of interactive model 522, or selected segments 562 of interactive model 560, at least initially (e.g., before adjustment by the EP). In some examples, target region map 648 is displayed in a distinct color. In some examples, a distinct hatching is used to display target region map 648, or any other suitable mechanism that allows the EP to easily determine the contours of target region map 648. Further, as displayed, a longitudinal axis 650 proceeds through an apex 608 of 3D structure image 602.

GUI 601 may, in some examples, display a reference character 680. The reference character 680 is displayed from a view according to an orientation of 3D structure image 602. For example, if the orientation of 3D structure image 602 is such that it is being displayed from an overhead view as the corresponding organ is positioned in the patient, then reference character 680 is displayed from an overhead view. This allows a medical professional, such as an EP, to easily determine from what view and/or orientation 3D structure image 602 is currently being displayed.

In some examples, GUI 601 includes one or more adjustment icons 655 that allow for an adjustment of 3D structure image 602. For example, adjustment icons 655 may allow for zoom in, zoom out, panning, and rotating functionalities.

With reference to FIG. 6B, GUI 601 may display one or more drag points, such as drag points 670A, 670B, that allow the EP to make adjustments to 3D structure image 602. For example, the EP may adjust longitudinal axis 650 by dragging drag point 670A to a new location. In response, GUI 400 adjust an orientation of scanned image 604 with respect to 3D segment model 606. Similarly, the EP may adjust target region map 648 my dragging drag point 670B to a new location. In some examples, GUI 601 allows for the creation, or removal, of drag points. For example, the EP may right-click on a drag point, such as drag point 670B, and select a “remove” option to remove the drag point. Likewise, the EP may right-click on a portion of 3D segment model 606, and select an “add” option to add a drag point.

FIG. 6C illustrates 3D structure image 602 after the EP provided input to rotate 3D structure image 602 clockwise around longitudinal axis 650 (e.g., using I/O device 203 to select one or more adjustment icons 655). In this example, drag point 670C may allow the EP to adjust an anterior interventricular groove 686 of 3D structure image 602.

Adjustment icons 655 may also allow the EP to display images of additional organs, such as organs that are adjacent to the organ identified by scanned image 604. For example, and with reference to FIG. 6D, the EP may select an adjustment icon 655 to display organ selection box 675, which allows the EP to select from one or more organs to display.

For example, and assuming the EP selects “lung” (e.g., “lung_r_p” for right lung, or “lung_l_p” for left lung) and “esophagus,” GUI 601 may display renderings (e.g., 3D renderings) of a first organ 685 (e.g., lung) and a second organ 687 (e.g., esophagus), as illustrated in FIG. 6E. The renderings may be 3D models pre-stored in database 116, for example. In other examples, the renderings are scanned images of the corresponding structure of the patient.

Further, in some examples, GUI 601 may further display distances 677 from the organ being treated (e.g., heart ventricle) to each of the other organs. In some examples, target recommendation computing device 104 determines the distances from a center of a scar location of the organ being treated to each of the other organs based on, for example, image data (e.g., image data 103) for the patient. In some examples, target recommendation computing device 104 determines the distances based on text extracted from report data, as described herein. For example, target recommendation computing device 104 may identify text describing the scar location, as well as text describing a location of another organ, and may determine the distance between the scar location and the other organ based on the locations.

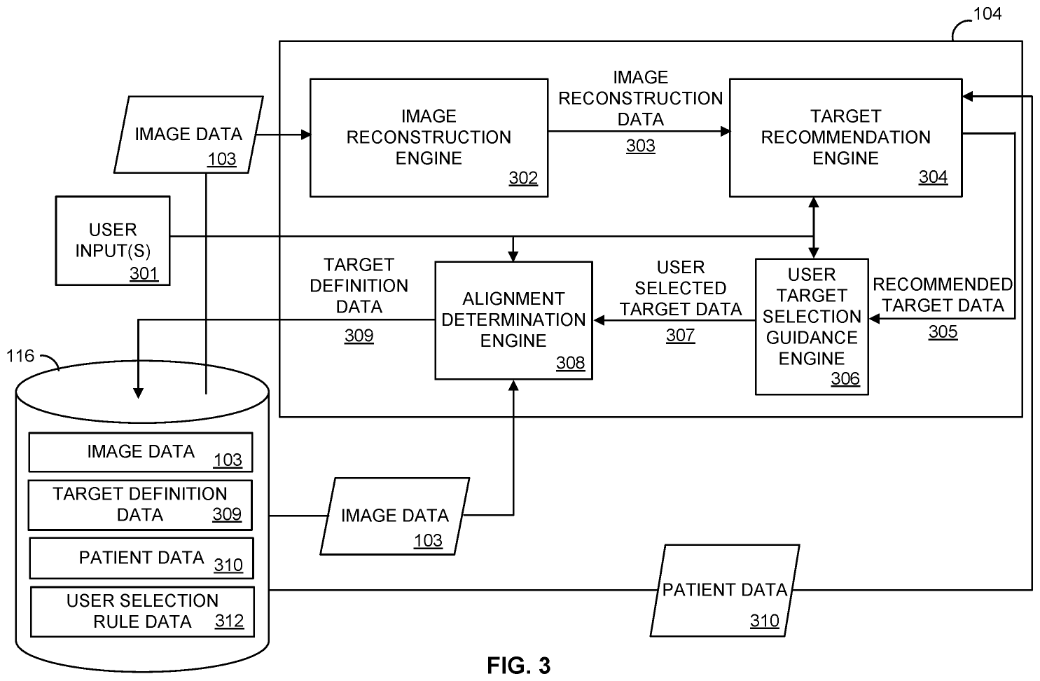

FIG. 3 illustrates exemplary portions of target recommendation computing device 104. In this example, target recommendation computing device 104 includes image reconstruction engine 302, target recommendation engine 304, user target selection guidance engine 306, and alignment determination engine 308. In some examples, one or more of image reconstruction engine 302, target recommendation engine 304, user target selection guidance engine 306, and alignment determination engine 308 may be implemented in hardware. In some examples, one or more of image reconstruction engine 302, target recommendation engine 304, user target selection guidance engine 306, and alignment determination engine 308 may be implemented as an executable program maintained in a tangible, non-transitory memory, such as instruction memory 207 of FIG. 2, that may be executed by one or processors, such as processor 201 of FIG. 2

In this example, one or more of target recommendation engine 304, user target selection guidance engine 306, and alignment determination engine 308 may receive one or more user inputs 301. For example, a medical professional may provide user input(s) 301 via input/output device 203, or via a touchscreen of display 206. User input(s) 301 may be received within a graphical user interface (GUI) provided by an executed application. Each of target recommendation engine 304, user target selection guidance engine 306, and alignment determination engine 308 may receive data from (e.g., user input(s) 301) the GUI, and may provide data to the GUI, such as data for display.

Image reconstruction engine 302 may obtain image data 103 for a patient from database 116. For example, image data 103 may be image data, such as CT image data or MR image data, captured with image scanning device 102 for the patient. Image reconstruction engine 302 may reconstruct an image based on the obtained image data 103. In some examples, the reconstructed image may be a 3-dimensional image of one or more organs of the patient. Image reconstruction engine 302 generates image reconstruction data 303 characterizing the reconstructed image, and provides image reconstruction data 303 to target recommendation engine 304.

Target recommendation engine 304 may perform operations to identify an initial target region for treatment based on the image reconstruction data 303. For example, target recommendation engine 304 may apply one or more trained machine learning processes to the image reconstruction data 303 to define the initial target region. The machine learning processes may be trained, using supervised or unsupervised learning, based on features generated from historical image scans, as described herein.

In some examples, target recommendation engine 304 determines the initial target area based on patient data 310 obtained from database 116 for the patient. Patient data 310 may characterize medical information about the patient, such as medical reports, previous procedures, current and previous conditions, diagnosis, current and previous treatments, and/or any other medical information. For example, patient data 310 may include report data characterizing medical professional findings and/or diagnosis of the patient. Target recommendation engine 304 may obtain the patient data 310 for the patient from database 116, and apply a text extracting process to the patient data 310 to identify text. Further, target recommendation engine 304 may apply a trained machine learning process to the text data as well as, in some examples, to the image reconstruction data 303, to determine the initial target area.

In some examples, target recommendation engine 304 applies one or more rules to the text data and/or the image reconstruction data 303 to determine the initial target area. A rule may associate, for example, one or more words of the text data with a first target area, and one or more different words of the text data with a second target area. Target recommendation engine 304 may determine, for example, if the extracted text includes any of the one or more words associated with the first target area, or the one or more different words associated with the second target area. Based on any corresponding words, target recommendation engine 304 may determine the initial target region as either the first target area or the second target area. In some examples, target recommendation engine 304 determines the initial target region to be the one with the most corresponding words.

Target recommendation engine 304 generates recommended target data 305 characterizing the determined initial target region. Recommended target data 305 may identify the initial target region within the reconstructed image, and additionally or alternatively, may identify a corresponding segment of a segment model, as described herein. Target recommendation engine 304 provides recommended target data 305 to user target selection guidance engine 306.

User target selection guidance engine 306 may allow a medical professional to update the initial target region. For example, user target selection guidance engine 306 may generate one or more GUIs, such as GUIs 520, 550, that allow the medical professional to change, update, or modify model segments corresponding to areas of treatment. In some instances, user target selection guidance engine 306 may display an interactive model, such as interactive model 522 or interactive model 560, and may receive an input (e.g., input 301) to select, or deselect, segments of the interactive model. User target selection guidance engine 306 updates the interactive model accordingly based on the input. Further, the one or more GUIs may further display sample images as described herein, such as within sample images 563 portion of GUI 550. In addition, the one or more GUIs may display segment-based notes, such as within a segment-based notes 564 portion of GUI 550, and may further provide alerts, such as within an alerts 565 portion of GUI 550, as described herein. User target selection guidance engine 306 may update the displayed segment-based notes and/or alerts as the medical professional selects and/or deselects segments of the interactive model. Further, user target selection guidance engine 306 may generate user selected target data 307 characterizing the selected segments, and may provide the user selected target data 307 to alignment determination engine 308.

Alignment determination engine 308 may perform operations to generate and provide for display a 3D model of the organ or portion thereof corresponding to the selected target data 307. Further, alignment determination engine 308 may receive image reconstruction data 303 characterizing the reconstructed image from image reconstruction engine 302, which in some examples may be a 3D image of the patient's heart ventricle. Alignment determination engine 308 may determine an alignment of the reconstructed image to the 3D model, and may superimpose the 3D model onto the reconstructed image according to the determined alignment to generate a 3D structure image. Alignment determination engine 308 may then provide the 3D structure image for display, such as for displaying on display 206.

Further, alignment determination engine 308 may receive user input(s) 301 identifying and characterizing adjustments to the 3D structure image. In response to the user input(s) 301, alignment determination engine 308 may adjust the 3D structure image accordingly. For example, alignment determination engine 308 may refine the alignment of the 3D model to the reconstructed image.

In some examples, alignment determination engine 308 determines whether each medical professional adjustment violates one or more predetermined rules (for example from the user selection rule data 312 in database 116). If an adjustment violates a rule, alignment determination engine 308 may cause the display of a pop-up message with a warning.

In some examples, alignment determination engine 308 receives one or more user input(s) 301 identifying a selection of one or more other organs that may be displayed in conjunction with the 3D structure image. In response, alignment determination engine 308 provides for display 3D models of such organs. In some examples, alignment determination engine 308 provides for display image data 103 of the patient's corresponding organs. In some examples, alignment determination engine 308 determines a distance between the organ being treated and each of the one or more other selected organs, and provides for display the determined distances.

In some examples, alignment determination engine 308 receives one or more user input(s) 301 identifying a pan or zoom action. In response, alignment determination engine 308 may pan or zoom across the 3D structure image. In some examples, alignment determination engine 308 receives one or more user input(s) 301 identifying the selection of a preconfigured selection for specific views of the 3D structure image. Alignment determination engine 308 may adjust the 3D structure image in accordance with the specific view selected, and may provide for display the adjusted 3D structure image.

Alignment determination engine 308 may generate target definition data 309 identifying and characterizing one or more of the refined 3D structure image and any other selected organs and determined distances, and may store target definition data 309 in database 116. In some examples, alignment determination engine 308 causes target recommendation computing device 104 to transmit the target definition data 309 to another computing device, such as treatment planning computing device 106, for treating the patient.

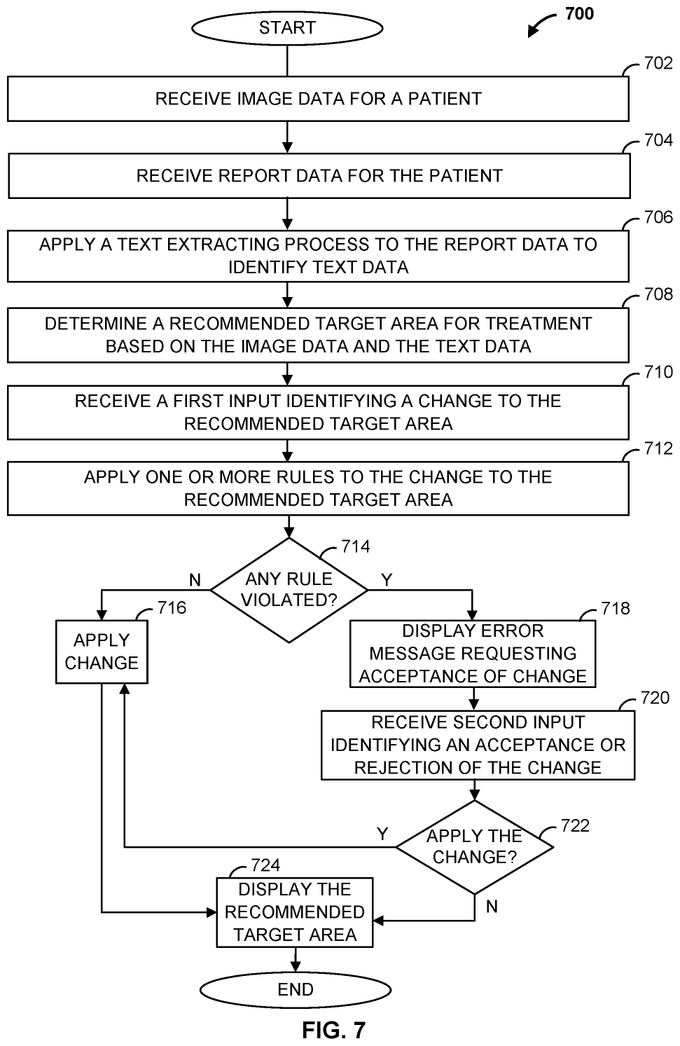

FIG. 7 illustrates a flowchart of an example method 700 that can be carried out by, for example, target recommendation computing device 104. Beginning at step 702, target recommendation computing device 104 receives image data for a patient. For example, target recommendation computing device 104 may obtain image data 103 from database 116, or may receive image data 103 from image scanning device 102. At step 704, target recommendation computing device 104 receives report data for the patient. For example, target recommendation computing device 104 may obtain patient data 310 for the patient from database 116. Further, and at step 706, target recommendation computing device 104 applies a text extracting process to the report data to identify text data. Target recommendation computing device 104 may apply any known text extracting process suitable to extract text from reports, for example.

At step 708, target recommendation computing device 104 determines a recommended target area for treatment based on the image data and the text data. For example, and as described herein, target recommendation computing device 104 may apply one or more trained machine learning processes, or may apply one or more rules, to the image data and the text data to determine the recommended target area. Further, and at step 710, target recommendation computing device 104 receives a first input identifying a change to the recommended target area. For example, a medical professional may select, or deselect, a segment of a corresponding interactive model (e.g., interactive model 522 or interactive model 560).

At step 712, target recommendation computing device 104 applies one or more rules to the change to the recommended target area and determines, at step 714, if any of the one or more rules are violated. If no rules are violated, the method proceeds to step 716 where target recommendation computing device 104 applies the change to the recommended target area (e.g., the corresponding model is saved within database 116 with the selected or deselected segment). The method then proceeds to step 724, where the recommended target area is displayed as updated. For example, the recommended target area may be displayed within a GUI.

If, however, at step 714, target recommendation computing device 104 determines that at least one rule is violated, the method proceeds to step 718, where target recommendation computing device 104 displays an error message requesting an acceptance of the change. For example, target recommendation computing device 104 may display a warning message asking the medical professional to verify the change, and may further display, for example, one or more alerts within an alert portion of the GUI, and/or one or more segment-based notes within a segment-based note portion of the GUI.

From step 718 the method proceeds to step 720 where target recommendation computing device 104 receives a second input, where the second input identifies an acceptance, or rejection, of the change. For example, the error displayed at step 718 may include an ACCEPT icon and a REJECT icon. The medical professional may select the ACCEPT icon to accept the change, or may, instead, select the REJECT icon to reject the change.

At step 722, target recommendation computing device 104 determines whether the change is accepted based on the second input. If the change is accepted, the method proceeds to step 716, where the change is applied. Otherwise, if the change is not accepted, the method proceeds to step 724, where the recommended target area is displayed without the change. The method then ends.

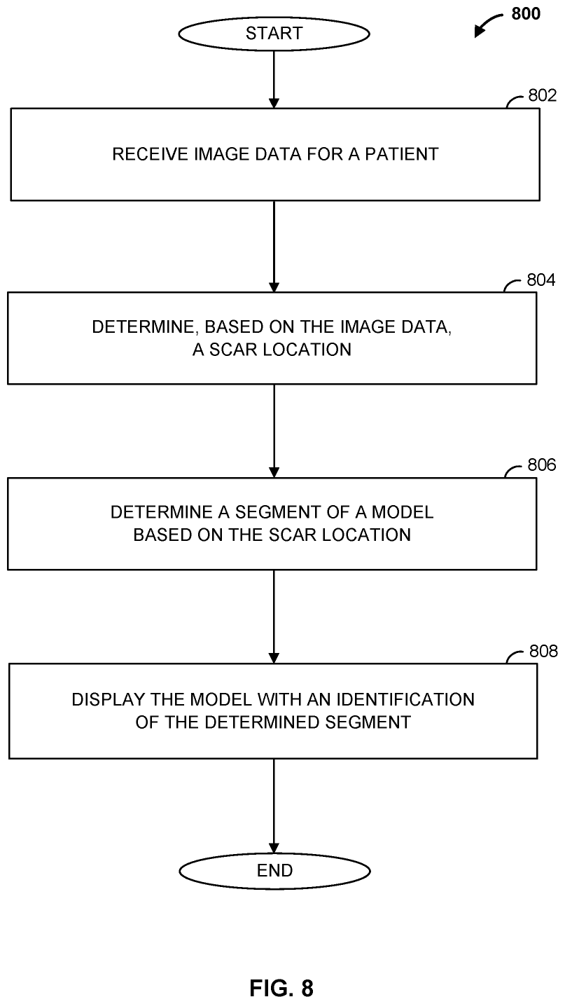

FIG. 8 is a flowchart of an example method 800 that can be carried out by, for example, target recommendation computing device 104. Beginning at step 802, image data for a patient is received. For example, target recommendation computing device 104 may obtain image data 103 from database 116, or may receive image data 103 from image scanning device 102. At step 804, target recommendation computing device 104 determines, based on the image data, a scar location. For example, and as described herein, target recommendation computing device 104 may determine the scar location based on applying one or more trained machine learning processes, and/or applying one or more rules, to the image data.

Further, and at step 806, target recommendation computing device 104 determines a segment of a model based on the scar location. For example, target recommendation computing device 104 may determine a segment of a segment model (e.g., a 17-segment model of a heart's ventricle) corresponding to the scar location. At step 808, target recommendation computing device 104 displays the segment model with an identification of the determined segment. For example, and as described herein, target recommendation computing device 104 may display the determined segment in a different color, or may highlight or hash the determined segment, or may identify the determined segment in any other suitable manner. The method then ends.

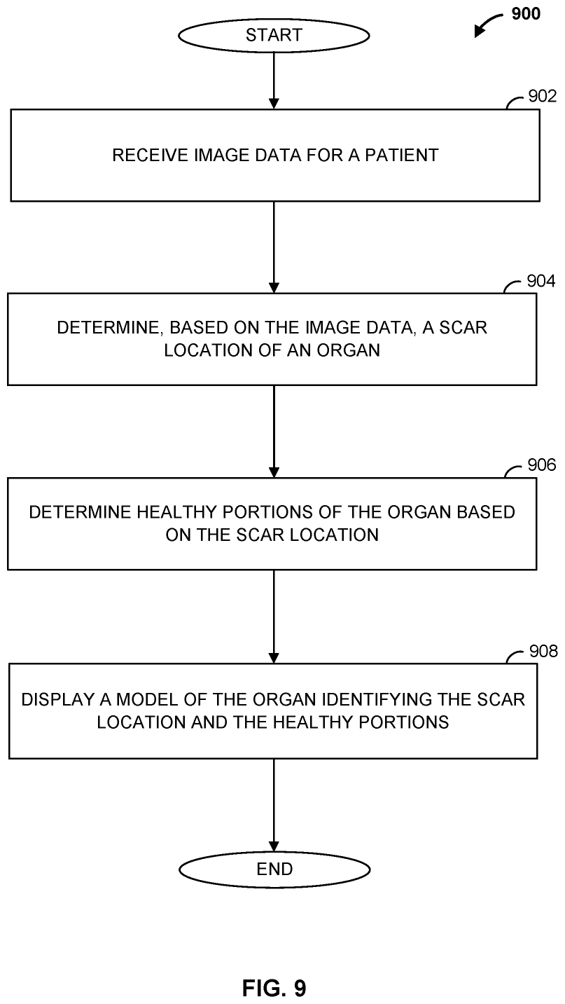

FIG. 9 is a flowchart of an example method 900 that can be carried out by, for example, target recommendation computing device 104. Beginning at step 902, image data for a patient is received. For example, target recommendation computing device 104 may obtain image data 103 from database 116, or may receive image data 103 from image scanning device 102. At step 904, target recommendation computing device 104 determines, based on the image data, a scar location of an organ. For example, and as described herein, target recommendation computing device 104 may determine the scar location of an organ based on applying one or more trained machine learning processes, and/or applying one or more rules, to the image data.

Further, and at step 906, target recommendation computing device 104 determines healthy portions of the organ based on the scar location. For example, and as described herein, target recommendation computing device 104 may identify portions of the organ that are at least a minimum distance from the scar location as healthy portions of the organ. In some examples, target recommendation computing device 104 applies one or more rules to text extracted from report data to determine the healthy portions.

At step 908, target recommendation computing device 104 displays the segment model with an identification of the scar location and the healthy portions of the organ. For example, and as described herein, target recommendation computing device 104 may display a segment model with segments corresponding to the scar location displayed differently than segments corresponding to healthy portions of the organ. For example, the segments corresponding to the scar location may be displayed in a different color, or highlighted or hashed differently, than the segments corresponding to the health portions of the organ. The method then ends.

In some examples, a computing device receives image data from one or more modalities for a patient. The computing device determines a recommended target area for treatment based on the image data, and determines one or more corresponding segments of a segment model based on the recommended target area. Further, the computing device displays the segment model identifying the determined one or more segments, and receives input data modifying the determined one or more segments. Based on the input data, the computing device updates the one or more segments, and generates target definition data characterizing the updated one or more segments. The computing device transmits the target definition data for treating the patient.

In some examples, a system includes a database, and a computing device communicatively coupled to the database. The computing device is configured to receive image data for an organ of a patient. The computing device is also configured to determine a recommended target area of the organ for treatment based on the image data. Further, the computing device is configured to generate recommended target data characterizing the recommended target area of the organ. The computing device is also configured to store the recommended target data in the database.

In some examples, the computing device is configured to receive report data characterizing medical findings of the patient, and determine the recommended target area based on the report data. In some examples, the computing device is configured to determine the recommended target area by applying a text extracting process to the report data to identify text within the report data. In some examples, the computing device is configured to determine the recommended target area based on applying a rule to the text.

In some examples, the computing device is configured to determine the recommended target area based on applying one or more machine learning models to the image data. In some examples, the computing device is configured to generate features based on historical image scans, and train the one or more machine learning models based on the generated features.

In some examples, the computing device is configured to transmit the recommended target data to a second computing device to treat the patient.

In some examples, a computing device is configured to receive a first input identifying a change to a recommended target area for treatment. The computing device is also configured to determine if a first rule is violated based on the change to the recommended target area for treatment. Further, the computing device is configured to provide for display an indication of whether the change is accepted based on determining if any of the one or more rules are violated.

In some examples, the computing device is configured to determine that the first rule is not violated, and update the recommended target area based on the change.

In some examples, the computing device is configured to determine that the first rule is violated, and provide for display an error message based on the violation.

In some examples, the computing device is configured to receive a second input, generate target data characterizing the recommended target area, and transmit the target data to a second computing device to treat a patient.

In some examples, the computing device is configured to display an interactive model on a graphical user interface, where the interactive model includes a plurality of segments, and where the first input identifies a selection of at least one segment of the plurality of segments. In some examples, the computing device is configured to display notes associated with the at least one segment of the plurality of segments. In some examples, the first rule is based on a particular combination of the plurality of segments. In some examples, the first rule is based on the selection of a maximum number of the plurality of segments.

In some examples, the computing device is configured to obtain image data for an organ of a patient, where the recommended target area for treatment is within the organ. The computing device is also configured to generate a segment model based on the organ, and display the segment model with an overlay of the image data.

In some examples, the computing device is configured to obtain image data for an organ of a patient, where the recommended target area for treatment is within the organ. The computing device is also configured to generate a segment model based on the organ, and display the image data with an overlay of the segment model.

In some examples, the computing device is configured to generate a first digital model of a type of the organ, and determine an alignment of the image data to the first digital model. The computing device is also configured to generate a second digital model comprising at least a portion of the scanned image and the first digital model. The computing device is further configured to store the second digital model in a data repository. In some examples, the computing device is further configured to provide the second digital model for display. In some examples, the computing device is further configured to receive a second input identifying an adjustment to the alignment of the image data to the first digital model. The computing device is also configured to adjust the second digital model based on the second input. The computing device is further configured to store the adjusted second digital model in the data repository.

In some examples, the computing device is configured to receive a second input identifying a treatment target area of the organ, and determine a corresponding portion of the second digital model based on the treatment target area of the organ. The computing device is also configured to regenerate the second digital model to identify the corresponding portion of the second digital model.

In some examples, a computing device is configured to receive image data for a patient. The computing device is also configured to determine a scar location of an organ based on the image data. Further, the computing device is also configured to determine a segment of a plurality of segments of a model of the organ based on the scar location. The computing device is also configured to display the model with an identification of the determined segment. For example, the computing device may display the determined segment in one color, and the remaining segments of the plurality of segments in another color. In some examples, the computing device is configured to display the model with an overlay of the image data. In some examples, the computing device is configured to display the image data with an overlay of the model.

In some examples, a computing device is configured to receive image data for a patient. The computing device is also configured to determine a scar location of an organ based on the image data. Further, the computing device is configured to determine healthy portions of the organ based on the scar location. The computing device is also configured to display a model of the organ with an identification of the scar location and the healthy portions. For example, the computing device may display the scar location of the organ in one color, and the healthy portions of the organ in another color.

In some examples, a computer-implemented method includes receiving image data for a patient. The method also includes determining, based on the received image data, a recommended target area for treatment. In some examples, the method also includes receiving report data for the patient. The method then includes determining the recommended target area for treatment based on the image data and the report data.

Further, the method includes receiving an input identifying a change to the recommended target area for treatment. The method also includes determining if any of one or more rules are violated based on the change to the recommended target area for treatment. The method further includes providing for display an indication of whether the change is accepted based on determining if any of the one or more rules are violated.

In some examples, a method includes receiving image data for an organ of a patient. The method also includes determining a recommended target area of the organ for treatment based on the image data. Further, the method includes generating recommended target data characterizing the recommended target area of the organ. The method also includes the recommended target data in a database.

In some examples, a computer-implemented method includes receiving a first input identifying a change to a recommended target area for treatment. The method also includes determining if a first rule is violated based on the change to the recommended target area for treatment. Further, the method includes providing for display an indication of whether the change is accepted based on determining if any of the one or more rules are violated.

In some examples, a computer-implemented method includes receiving image data for a patient. The method also includes determining a scar location of an organ based on the image data. Further, the method includes determining a segment of a plurality of segments of a model of the organ based on the scar location. The method also includes displaying the model with an identification of the determined segment. In some examples, the method includes displaying the model with an overlay of the image data. In some examples, the method includes displaying the image data with an overlay of the model.

In some examples, a computer-implemented method includes receiving image data for a patient. The method includes determining a scar location of an organ based on the image data. Further, the method includes determining healthy portions of the organ based on the scar location. The method also includes displaying a model of the organ with an identification of the scar location and the healthy portions.

In some examples, a non-transitory computer readable medium storing instructions that, when executed by at least one processor, cause the at least one processor to perform operations including receiving image data for a patient. The operations also include determining, based on the received image data, a recommended target area for treatment. In some examples, the operations also include receiving report data for the patient. The operations then include determining the recommended target area for treatment based on the image data and the report data.

Further, the operations include receiving an input identifying a change to the recommended target area for treatment. The operations also include determining if any of one or more rules are violated based on the change to the recommended target area for treatment. The operations further include providing for display an indication of whether the change is accepted based on determining if any of the one or more rules are violated.

In some examples, a non-transitory computer readable medium storing instructions that, when executed by at least one processor, cause the at least one processor to perform operations including receiving image data for an organ of a patient. The operations also include determining a recommended target area of the organ for treatment based on the image data. Further, the operations include generating recommended target data characterizing the recommended target area of the organ. The operations also include the recommended target data in a database.

In some examples, a non-transitory computer readable medium storing instructions that, when executed by at least one processor, cause the at least one processor to perform operations including receiving a first input identifying a change to a recommended target area for treatment. The operations also include determining if a first rule is violated based on the change to the recommended target area for treatment. Further, the operations include providing for display an indication of whether the change is accepted based on determining if any of the one or more rules are violated.

In some examples, a non-transitory computer readable medium storing instructions that, when executed by at least one processor, cause the at least one processor to perform operations including receiving image data for a patient. The operations also include determining a scar location of an organ based on the image data. Further, the operations include determining a segment of a plurality of segments of a model of the organ based on the scar location. The operations also include displaying the model with an identification of the determined segment. In some examples, the operations include displaying the model with an overlay of the image data. In some examples, the operations include displaying the image data with an overlay of the model.

In some examples, a non-transitory computer readable medium storing instructions that, when executed by at least one processor, cause the at least one processor to perform operations including receiving image data for a patient. The operations include determining a scar location of an organ based on the image data. Further, the operations include determining healthy portions of the organ based on the scar location. The operations also include displaying a model of the organ with an identification of the scar location and the healthy portions.

In some examples, a computer-implemented method includes a means for receiving image data for a patient. The method also includes a means for determining, based on the received image data, a recommended target area for treatment. In some examples, the method also includes a means for receiving report data for the patient. The method then includes a means for determining the recommended target area for treatment based on the image data and the report data.

Further, the method includes a means for receiving an input identifying a change to the recommended target area for treatment. The method also includes a means for determining if any of one or more rules are violated based on the change to the recommended target area for treatment. The method further includes a means for providing for display an indication of whether the change is accepted based on determining if any of the one or more rules are violated.

In some examples, a computer-implemented method includes a means for receiving image data for an organ of a patient. The method also includes a means for determining a recommended target area of the organ for treatment based on the image data. Further, the method includes a means for generating recommended target data characterizing the recommended target area of the organ. The method also includes a means for storing the recommended target data in a database.

In some examples, a computer-implemented method includes a means for receiving a first input identifying a change to a recommended target area for treatment. The method also includes a means for determining if a first rule is violated based on the change to the recommended target area for treatment. Further, the method includes a means for providing for display an indication of whether the change is accepted based on determining if any of the one or more rules are violated.

In some examples, a computer-implemented method includes a means for receiving image data for a patient. The method also includes a means for determining a scar location of an organ based on the image data. Further, the method includes a means for determining a segment of a plurality of segments of a model of the organ based on the scar location. The method also includes a means for displaying the model with an identification of the determined segment. In some examples, the method includes a means for displaying the model with an overlay of the image data. In some examples, the method includes a means for displaying the image data with an overlay of the model.

In some examples, a computer-implemented method includes a means for receiving image data for a patient. The method includes a means for determining a scar location of an organ based on the image data. Further, the method includes a means for determining healthy portions of the organ based on the scar location. The method also includes a means for displaying a model of the organ with an identification of the scar location and the healthy portions.

Although the methods described above are with reference to the illustrated flowcharts, it will be appreciated that many other ways of performing the acts associated with the methods can be used. For example, the order of some operations may be changed, and some of the operations described may be optional.

In addition, the methods and system described herein can be at least partially embodied in the form of computer-implemented processes and apparatus for practicing those processes. The disclosed methods may also be at least partially embodied in the form of tangible, non-transitory machine-readable storage media encoded with computer program code. For example, the steps of the methods can be embodied in hardware, in executable instructions executed by a processor (e.g., software), or a combination of the two. The media may include, for example, RAMs, ROMs, CD-ROMs, DVD-ROMs, BD-ROMs, hard disk drives, flash memories, or any other non-transitory machine-readable storage medium. When the computer program code is loaded into and executed by a computer, the computer becomes an apparatus for practicing the method. The methods may also be at least partially embodied in the form of a computer into which computer program code is loaded or executed, such that, the computer becomes a special purpose computer for practicing the methods. When implemented on a general-purpose processor, the computer program code segments configure the processor to create specific logic circuits. The methods may alternatively be at least partially embodied in application specific integrated circuits for performing the methods.

The foregoing is provided for purposes of illustrating, explaining, and describing embodiments of these disclosures. Modifications and adaptations to these embodiments will be apparent to those skilled in the art and may be made without departing from the scope or spirit of these disclosures.

1. A system comprising:
a computing device configured to:
receive a first input identifying a change to a target area for treatment;
determine if a first rule is violated based on the change to the target area for treatment; and
provide for display an indication of whether the change is accepted based on determining if any of the one or more rules are violated.

2. The system of claim 1, wherein the computing device is configured to:
determine that the first rule is not violated; and
update the target area based on the change.

3. The system of claim 1, wherein the computing device is configured to:
determine that the first rule is violated; and
provide for display an error message based on the violation.

4. The system of claim 1, wherein the image data is at least one of magnetic resonance image data and computed tomography image data.

5. The system of claim 1, wherein the computing device is configured to:
receive a second input;
generate target data characterizing the target area; and
transmit the target data to a second computing device to treat a patient.

6. The system of claim 1, wherein the computing device is configured to display an interactive model on a graphical user interface, wherein the interactive model includes a plurality of segments, and wherein the first input identifies a selection of at least one segment of the plurality of segments.

7. The system of claim 6, wherein the computing device is configured to display notes associated with the at least one segment of the plurality of segments.

8. The system of claim 6 or claim 7, wherein the first rule is based on a particular combination of the plurality of segments.

9. The system of claim 6, wherein the first rule is based on the selection of a maximum number of the plurality of segments.

10. The system of claim 1, wherein the computing device is configured to:
obtain image data for an organ of a patient, wherein the target area for treatment is within the organ;
generate a segment model based on the organ; and
display the segment model with an overlay of the image data.

11. The system of claim 1, wherein the computing device is configured to:
obtain image data for an organ of a patient, wherein the target area for treatment is within the organ;
generate a segment model based on the organ; and
display the image data with an overlay of the segment model.

12. The system of claim 1, wherein the computing device is configured to:
generate a first digital model of a type of the organ;
determine an alignment of the image data to the first digital model;
generate a second digital model comprising at least a portion of the scanned image and the first digital model; and
store the second digital model in a data repository.

13. The system of claim 12, wherein the computing device is further configured to provide the second digital model for display.

14. The system of claim 12, wherein the computing device is further configured to:
receive a second input identifying an adjustment to the alignment of the image data to the first digital model;
adjust the second digital model based on the second input; and
store the adjusted second digital model in the data repository.

15. The system of claim 1, wherein the computing device is further configured to:
receive a second input identifying a treatment target area of the organ;
determine a corresponding portion of the second digital model based on the treatment target area of the organ; and
regenerate the second digital model to identify the corresponding portion.

16. A computer-implemented method comprising:
receiving a first input identifying a change to a target area for treatment;
determining if a first rule is violated based on the change to the target area for treatment; and
providing for display an indication of whether the change is accepted based on determining if any of the one or more rules are violated.

17. The computer-implemented method of claim 16 comprising:
determining that the first rule is not violated; and
updating the target area based on the change.

18. The computer-implemented method of claim 16 comprising:
determining that the first rule is violated; and
providing for display an error message based on the violation.

19. A non-transitory computer readable medium storing instructions that, when executed by at least one processor, cause the at least one processor to perform operations comprising:
receiving a first input identifying a change to a target area for treatment;
determining if a first rule is violated based on the change to the target area for treatment; and
providing for display an indication of whether the change is accepted based on determining if any of the one or more rules are violated.

20. The non-transitory computer readable medium of claim 19 wherein the operations further comprise at least one of:
determining that the first rule is not violated; and
updating the target area based on the change; or
determining that the first rule is violated; and
providing for display an error message based on the violation
